(19) United States
(12) Patent Application Publication
(10) Pub. No.: US 2026/0021264 A1
(43) Pub. Date: Jan. 22, 2026

(54) NOISE-REDUCING AIR PASSAGE DEVICE FOR USE IN A PAP MACHINE

(71) Applicant: WALLENBERG UNION LLC, Newark, DE (US)

(72) Inventor: David LUO, Newark, DE (US)

(21) Appl. No.: 18/778,477

(22) Filed: Jul. 19, 2024

(51) Int. Cl. A61M 16/00

(52) U.S. Cl. CPC A61M 16/0066; A61M 16/0003; A61M 2202/0007; A61M 2202/02; A61M 2205/0216; A61M 2205/3331; A61M 2205/42; A61M 2206/14; F04D 29/663; F04D 29/667

(57) ABSTRACT

A noise-reducing air passage device, configured to be used in a PAP machine. The device includes several parts, including a casing with at least one outlet, at least one inlet, an inner wall, and an outer wall. The casing includes at least two parts that form a seal when the device is in operation, such that a gas passage for air flow is formed by the space enclosed by the inner wall of the casing. The inner wall forms at least one chamber within the gas passage, and a blower is provided within the chamber. The blower is configured to pressurize gas that enters the chamber and deliver the pressurized gas to the outlet of the casing. The device can also include a ventilation component, an inlet pipe and an outlet pipe.

### TECHNICAL FIELD

This disclosure pertains to a noise-reducing air passage device for use in a PAP (Positive Airway Pressure) machine. It involves a gas passage formed by a casing and internal noise-reducing components, as well as the placement of a blower.

### BACKGROUND

Sleep accounts for about one-third of a human's lifespan, during which sleep-related respiratory disorders, which can disrupt normal breathing patterns, are particularly prone to occur. These disorders can lead to symptoms like apnea, breathing difficulties, or other respiratory issues, substantially interfering with normal sleep. Common sleep-related respiratory diseases include snoring, Sleep Apena Syndrome (SAS), hypopnea, hypoventilation, sleep-related hypoxemia, and other rarer types. Furthermore, a patient may suffer from more than one type of sleep-related respiratory disorder. For instance, over 90% of patients with obesity hypoventilation syndrome also suffer from Obstructive Sleep Apnea (OSA); likewise, patients with Chronic Obstructive Pulmonary Disease (COPD) may experience hypoxemia during sleep. The incidence of complications is higher in patients with multiple sleep-related respiratory diseases, and the symptoms are more severe. Thus, addressing sleep-related respiratory diseases is critical. Early identification and treatment of predisposed patients are crucial for improving sleep quality, reducing the risk of complications, and enhancing quality of life.

SAS is one of the most common sleep-related respiratory disorders and includes both obstructive and central sleep apnea syndromes. Obstructive Sleep Apnea Syndrome (OSAS) typically refers to partial or complete blockage of the airway during sleep, causing pauses in breathing or shallow breathing. Central Sleep Apnea Syndrome (CSAS), on the other hand, occurs when the brain fails to send adequate signals to control the respiratory muscles, leading to pauses in breathing. Treatment approaches differ for these two types of SAS. For patients with OSAS, the most common treatments include using home ventilators, lifestyle changes, or oral corrective surgeries; for CSAS, treatment focuses on addressing underlying central nervous system anomalies. OSA is more common than central sleep apnea and has a relatively high prevalence, especially among adults.

When treating patients with OSA using home ventilators, the principle involves providing a continuous positive airflow with the ventilator to keep the upper airway clear. The core of ventilator therapy is the use of a blower to generate pressurized airflow, which is delivered to the patient's nose or mouth via the tube connected to the ventilator. This keeps the airway under positive pressure to prevent airway collapse or closure during sleep, thus avoiding apnea and airway blockages. Compared to traditional treatment methods, using home ventilators for treating OSA offers better convenience and comfort. It enables treatment at home, reduces the frequency of hospital visits, and enhances the accessibility and convenience of treatment. Moreover, existing home ventilators often include auxiliary functions, such as heated humidifiers to improve comfort, and the airflow can be adjusted based on the patient's own breathing. This allows for adaptation to the diverse needs of different patients. Increasingly, ventilators on the market also feature smart functions such as data recording and treatment effect monitoring; ventilators can manage and adjust settings effectively based on data collected from different patients' usage, optimizing experience for the next use. These auxiliary features and data monitoring systems help patients better understand their treatment effects and make necessary adjustments, ensuring the effectiveness and continuity of treatment, and enhancing the personalization and precision of therapy.

In summary, using home ventilators to treat OSA has several advantages, including effective treatment, convenience, comfort, personalized adjustment, and intelligent monitoring. Therefore, home ventilators have become one of the common treatment methods for patients with OSA, playing a positive role in improving patients' sleep quality and overall health.

### SUMMARY

The objective of this disclosure is to provide a novel noise-reducing air passage device for use in a PAP machine, utilizing internal noise-reducing components combined with a casing structure. This design is easier to manufacture and can quickly adapt to market needs, achieving regulatory noise levels without the use of foam. Patients can choose between foam-filled and foam-free noise-reducing air passage devices based on their tolerance to noise. Foam-free air passage devices ensure patient health and safety, while those containing foam can achieve quieter operation compared to existing products, overcoming limitations present in current technology. It provides a more effective solution with broader application scenarios, supplying continuous positive air pressure to treat sleep-related breathing disorders in a safer manner.

A noise-reducing air passage device for use in a PAP machine, configured to generate and deliver pressurized gas to an airway of a patient, is provided in this disclosure. The noise-reducing air passage device includes a casing, a gas passage and a blower. The casing includes at least two parts and has at least one outlet, at least one inlet, an inner wall, and an outer wall. The gas passage includes at least one chamber configured to provide a space for gas accumulation and flow. The gas passage is formed by space enclosed by the inner wall of the casing, and a total volume of the at least one chamber is between 3 to 18 times a volume of the blower. The blower includes an intake port to receive gas and an exhaust port to allow gas to exit, provided within one of the at least one chamber and configured to pressurize gas that enters the at least one chamber and deliver the pressurized gas to the at least one outlet of the casing. At least one inlet pipe is provided at and connectable to the at least one inlet of the casing, and the at least inlet pipe is configured to deliver gas from an external environment into the at least one chamber within the casing. The at least one outlet and the at least one inlet are not provided on a same wall of the casing, and the at least one chamber does not include foam.

In one embodiment, the at least one inlet pipe is configured to be integrally formed with the casing.

In one embodiment, a distance between the intake port of the blower and the inner wall of the casing is at least 5 mm.

In one embodiment, an axis of the at least one inlet pipe is parallel or perpendicular to an axis of the intake port of the blower.

In one embodiment, an outlet pipe is provided at the at least one outlet and communicates with the exhaust port of the blower.

In one embodiment, the casing forms a part of the PAP machine.

In another embodiment, a noise-reducing air passage device for use in a PAP machine, configured to generate and deliver pressurized gas to an airway of a patient, is provided in this disclosure. The noise-reducing air passage device includes a casing, a gas passage and a blower. The casing includes at least two parts, having at least one outlet, at least one inlet, an inner wall, and an outer wall. The gas passage includes at least two chambers configured to provide a space for gas accumulation and flow. The gas passage is formed by space enclosed by the inner wall of the casing, a total volume of the at least two chambers is between 3 to 18 times a volume of a blower. The blower includes an intake port to receive gas and an exhaust port to allow gas to exit, provided within one of the at least two chambers and configured to pressurize gas that enters the at least two chambers and deliver the pressurized gas to the at least one outlet of the casing. At least one inlet pipe is provided at and connectable to the at least one inlet of the casing, and the at least one inlet pipe is configured to deliver gas from an external environment into the at least two chambers within the casing. And the at least two chambers do not include foam.

In one embodiment, the inner wall of the casing has a silicone layer connectable to the inner wall, and the silicone layer is configured to reduce noise within the gas passage.

In one embodiment, the at least one inlet pipe is provided at an edge portion of the casing, and the at least one inlet pipe and the at least one outlet of the casing are not on a same wall of the casing.

In one embodiment, the at least one inlet pipe is tapered.

In one embodiment, at least part of a distance between the intake port of the blower and its opposing inner wall of the casing is at least 2 mm.

In one embodiment, the casing forms a part of the PAP machine.

In yet another embodiment, a noise-reducing air passage device for use in a PAP machine, configured to generate and deliver pressurized gas to an airway of a patient, is provided in this disclosure. The noise-reducing air passage device includes a casing, a gas passage and a blower. The casing includes at least two parts, having at least one outlet, at least one inlet, an inner wall, and an outer wall. The gas passage includes at least one chamber configured to provide a space for gas accumulation and flow, the gas passage is formed by space enclosed by the inner wall of the casing, a total volume of the at least one chamber is between 3 to 18 times a volume of a blower. The blower includes an intake port to receive gas and an exhaust port to allow gas to exit, provided within one of the at least one chamber and configured to pressurize gas that enters the at least one chamber and deliver the pressurized gas to the at least one outlet of the casing. At least one inlet pipe is provided at and connectable to the at least one inlet of the casing, and the at least one inlet pipe is configured to deliver gas from an external environment into the at least one chamber within the casing. An axis of the intake port of the blower is non-parallel to an axis of the at least one inlet of the casing.

In one embodiment, the inner wall of the casing has a silicone layer connectable to the inner wall, and the silicone layer is configured to reduce noise within the gas passage.

In one embodiment, the inner wall is configured to form at least two chambers for gas accumulation within the gas passage.

In one embodiment, a main path of the gas within the gas passage is an airflow path, and the inner wall opposite the airflow path has an arcuate surface.

In one embodiment, at least part of a distance between the intake port of the blower and its opposing inner wall of the casing is at least 2 mm.

In one embodiment, the casing forms a part of the PAP machine.

In one embodiment, the at least one chamber does not include foam.

In another embodiment, a noise-reducing air passage device for use in a PAP machine, configured to generate and deliver pressurized gas to an airway of a patient, is provided in this disclosure. The noise-reducing air passage device includes a casing, a gas passage and a blower. The casing includes at least two parts, having at least one outlet, at least one inlet, an inner wall, and an outer wall. The gas passage includes at least two chambers configured to provide a space for gas accumulation and flow, the gas passage is formed by space enclosed by the inner wall of the casing, a total volume of the at least two chambers is between 3 to 18 times a volume of a blower. The blower includes an intake port to receive gas and an exhaust port to allow gas to exit, provided within one of the at least two chambers and configured to pressurize gas that enters the at least two chambers and deliver the pressurized gas to the at least one outlet of the casing. The noise-reducing air passage device further includes at least one ventilation component that is provided within the gas passage, configured to communicate with the at least two chambers and provide a channel for gas to flow from one chamber to another. A main path of the gas within the gas passage is an airflow path, the airflow path is configured to have displacements along the x-axis, y-axis, and z-axis in a three-dimensional Cartesian coordinate system, and a total length of the airflow path is greater than 20 cm, and an area of the at least one inlet of the casing is greater than or equal to an area of the intake port of the blower.

In one embodiment, the at least two chambers include a first chamber and a second chamber, the blower is provided within the first chamber and the intake port of the blower communicates with the second chamber.

In one embodiment, the second chamber is smaller than the first chamber.

In one embodiment, the ventilation component includes an intake end and an exhaust end, and a distance between the exhaust end and its opposing inner wall of the casing is at least 3.5 mm.

In one embodiment, an outlet pipe is provided at the at least one outlet and communicates with the exhaust port of the blower.

In one embodiment, the casing forms a part of the PAP machine.

The implementation of the noise-reducing air passage device provided by this disclosure has at least the following beneficial effects:

1. Ventilators must achieve a noise level of 30 dB or less for marketing approval in FDA's regulations. Using foam for noise reduction is currently the simplest method. Foam materials, which are readily available and easy to manufacture, can convert noise into minute amounts of energy due to their unique porous structure and material properties. Indeed, using foam for noise reduction achieves effective noise mitigation and the use of foam within the air passage device is the most straightforward and common method to meet regulatory noise levels.

Consequently, nearly all PAP machines on the market contain foam within the gas passage device for noise reduction. However, foam can easily cause health issues for several reasons: 1) Foam materials are soft and have a relatively loose surface, which can wear or peel off during use, releasing particles. Once these particles are released, they can enter the patient's respiratory tract with the airflow, causing irritation to the respiratory system. This may lead to respiratory issues, resulting in symptoms such as sore throat and coughing, especially in individuals who already suffer from asthma or Chronic Obstructive Pulmonary Disease (COPD). 2) Moreover, foam is usually made from synthetic materials that may contain chemical additives. These chemicals can gradually release into the air as the foam ages. In some cases, if foam particles carry harmful microorganisms, they could cause potential infections, particularly in individuals with compromised immune systems. Foam particles may also trigger allergic reactions, including sneezing, flu-like symptoms, and eye irritation. 3) Additionally, foam used over long periods can accumulate dust, bacteria, and other contaminants. This is particularly concerning in PAP machines, where the foam can draw pollutants from the air into the machine, fostering bacterial growth and increasing the risk of infection.

Therefore, the disclosure particularly focuses on the safety and reliability of air passage devices of the ventilator during design, involving a series of improvements, including a foam-free design for the noise-reducing air passage device, to reduce potential health risks for patients using PAP machines. The gas passage and filtration system are designed foam-free or using easily replaceable foam to reduce these potential health risks. For health and safety, the gas passage in the disclosure is designed without foam. Since there is no foam within the gas passage, the likelihood of accumulating small foreign particles is reduced, helping to maintain the cleanliness of the gas passage. More importantly, in the absence of foam, the breathable air will not be affected by the tiny residues of the foam, reducing the number of particles that patients might inhale or come into contact with, thus ensuring safety during use. This is especially important for patients who use the device long-term, as it helps to reduce potential respiratory issues. Additionally, some patients may be allergic to particles from materials such as foam, and the foam-free design reduces the risk associated with allergic reactions. This is particularly significant for patients who are allergic to foam materials or sensitive to chemically treated materials. This product has undergone multiple tests to verify that the foam-free noise-reducing air passage device improves the safety and comfort of patients using the device.

2. The design of various noise-reducing components and the internal space structure of the noise-reducing air passage device ensures compliance with regulatory noise levels, even in the absence of foam. 1) This disclosure uses various types of noise-reducing components and structures, which combined, form an efficient noise-reduction system achieving significant noise mitigation. a. The internal space structure of the noise-reducing air passage device is configured to extend the airflow path within the gas passage. By lengthening the airflow path, the residence time of the gas within the gas passage increases, helping to reduce the speed of the airflow and the potential for turbulence or vortices, thereby lowering the noise level. A longer airflow path also means that the gas travels a greater distance within the gas passage, allowing noise to attenuate further during transmission. Therefore, extending the airflow path can increase the attenuation distance of noise, making it weaker by the time it reaches the designated position. b. The disclosure also introduces a ventilation component within the gas passage for noise reduction, an innovative structure not found in existing PAP machines' gas passages. The ventilation component effectively reduces turbulence and vortices in the airflow, thereby lowering noise levels. Additionally, the ventilation component offers advantages over existing noise-reducing components in PAP machines on the market. The ventilation component is simple in structure, integrally molded from a single material, eliminating unnecessary steps in manufacturing and installation, thereby reducing costs. Moreover, it can be individually optimized with different forms of microstructural variations, including changes to its baffle design. Due to its simple structure, the ventilation component can be installed at various positions within the gas passage for noise reduction. The noise-reducing air passage device can also include a combination of ventilation components of the same or different forms, providing greater flexibility in its application across different noise-reducing air passage devices. The core of the ventilation component lies in its internal structural design, allowing it to maintain its structure while adapting its external shape to fit different types of noise-reducing air passage devices or be placed in various positions within the device, such as inside the inlet pipe. This flexible design allows the ventilation component to be customized for specific applications to achieve better noise reduction. Regardless of the type of noise-reducing air passage devices, the ventilation component can maintain its noise-reducing performance, providing an efficient noise control solution for PAP machines. c. An inlet pipe is set at the inlet. Through the inlet pipe, external air can enter the chamber in a more orderly manner, reducing potential turbulence and noise generated when the airflow enters the chamber. Additionally, the inlet pipe stabilizes the airflow path within the chamber, reducing resistance as the air enters, further decreasing noise. The inlet pipe also enhances the efficiency of air transmission into the chamber, improving airflow characteristics and enhancing the overall performance of the PAP machine. d. The arrangement of the inlet and outlet not being on the same wall of the casing typically helps in noise reduction. Normally, the inlet is one of the primary sources of noise inside a PAP machine. Placing the inlet and outlet on different walls of the casing can prevent noise accumulation at the inlet, thus reducing noise levels at the inlet and enhancing the overall noise reduction effect. The combination of the above noise-reducing structures and components allows the noise-reducing air passage device to meet regulatory noise levels without foam and is a more effective and safer new noise reduction method. 2) By employing the above design of the noise-reducing air passage device, the disclosure achieves regulatory noise levels without foam.

When foam is introduced within the noise-reducing air passage device provided by this disclosure, it can achieve even higher noise reduction levels compared to the foam-free ones. Therefore, while ensuring safety, the introduction of foam or other soundproofing materials (such as silicone, gel, etc.) within the gas passage increases the absorption and isolation of noise in the airflow, further reducing the propagation and impact of noise. Compared to the foam-free situation, a noise-reducing air passage device with foam can provide a quieter environment, offering patients a more comfortable experience. This design retains the noise reduction advantages of the foam-free condition while further enhancing the noise reduction effect, achieving or even surpassing the highest noise reduction levels of existing PAP machines on the market, thus meeting the demand for higher noise reduction performance.

3. The noise-reducing air passage device provided by this disclosure has a simple structure and modular advantages, making it a cost-effective solution for manufacturers. The simple structure of the ventilation component allows for easy assembly or standalone use, enhancing efficiency during manufacturing, assembly, and maintenance. The modular approach enables different parts to be independently manufactured and upgraded, improving production efficiency and reducing costs. Manufacturers can customize the noise-reducing air passage devices by assembling components to meet various patient needs or product specifications. This economic design reduces manufacturing costs and offers patients more flexible and affordable options. Since the primary goal of this disclosure is to achieve regulatory noise levels without foam, there is no need to consider the layout and installation of foam. Consequently, noise-reducing air passages without foam have a simpler internal structure compared to existing market options. The simplified structure enhances the efficiency of manufacturing and assembly processes, reducing production costs and cycles.

### BRIEF DESCRIPTION OF THE DRAWINGS

FIG. 1 is a three-dimensional schematic diagram of a noise-reducing air passage device in accordance with one embodiment;

FIG. 2 is a three-dimensional schematic diagram of the casing of a noise-reducing air passage device in accordance with one embodiment;

FIG. 3 is an exploded view of the casing of a noise-reducing air passage device in accordance with one embodiment;

FIG. 4 is a three-dimensional schematic diagram of the blower of a noise-reducing air passage device in accordance with one embodiment;

FIG. 5 is an exploded view of the structure of a noise-reducing air passage device in accordance with one embodiment;

FIG. 6 is a schematic diagram of the airflow path of a noise-reducing air passage device in accordance with one embodiment;

FIG. 7 is a test scenario diagram of a noise-reducing air passage device in accordance with one embodiment;

FIG. 8 is a top view of the internal structure of a noise-reducing air passage device in accordance with one embodiment;

FIG. 9 is a schematic diagram of the ventilation component of a noise-reducing air passage device in accordance with one embodiment;

FIG. 10 is a sectional view of a noise-reducing air passage device in accordance with one embodiment;

FIG. 11 is a schematic diagram of the airflow passing through the ventilation component of a noise-reducing air passage device in accordance with one embodiment;

FIG. 12 is a schematic diagram of another form of the ventilation component of a noise-reducing air passage device in accordance with one embodiment;

FIG. 13 is a schematic diagram of the distance between the exhaust end of the ventilation component and its opposing inner wall of the casing in a noise-reducing air passage device in accordance with one embodiment;

FIG. 14 is a schematic diagram of the taper of the inlet pipe in a noise-reducing air passage device in accordance with one embodiment;

FIG. 15 is a schematic diagram showing the axis of the inlet pipe parallel to the axis of the blower intake port in a noise-reducing air passage device in accordance with one embodiment;

FIG. 16 is a schematic diagram showing the axis of the inlet pipe perpendicular to the axis of the blower intake port in a noise-reducing air passage device in accordance with one embodiment;

FIG. 17 is a schematic diagram of the distance between the blower intake port and its opposing wall of the casing in a noise-reducing air passage device in accordance with one embodiment;

FIG. 18 is a schematic diagram of a noise-reducing air passage device placed within a coordinate system in accordance with one embodiment;

FIG. 19 is a schematic diagram of the total length of the airflow path in a noise-reducing air passage device in accordance with one embodiment;

FIG. 20 is a schematic diagram of the inlet pipe within the chamber in a noise-reducing air passage device in accordance with one embodiment;

FIG. 21 is a schematic diagram of the inlet and outlet being provided on the same wall of the casing in a noise-reducing air passage device in accordance with one embodiment;

FIG. 22 is a three-dimensional schematic diagram of the casing of a noise-reducing air passage device forming part of a PAP machine in accordance with one embodiment;

FIG. 23 is a sectional view of the casing of a noise-reducing air passage device forming part of a PAP machine in accordance with one embodiment;

FIG. 24 is a schematic diagram of the chamber with foam of a noise-reducing air passage device in accordance with one embodiment;

FIG. 25 is a schematic diagram of the inner wall of the casing with silicone of a noise-reducing air passage device in accordance with one embodiment;

FIG. 26 is a three-dimensional diagram of a noise-reducing air passage device with another form of the internal structure in accordance with one embodiment;

FIG. 27 is a three-dimensional diagram of a noise-reducing air passage device with another form of the internal structure in accordance with one embodiment;

FIG. 28 is a three-dimensional diagram of a noise-reducing air passage device with another form of the internal structure in accordance with one embodiment;

FIG. 29 is a schematic diagram analyzing the internal airflow direction of a noise-reducing air passage device in accordance with one embodiment.

### DETAILED DESCRIPTION

To facilitate the understanding of the disclosure, a more comprehensive description will be provided with reference to the relevant drawings. The drawings illustrate typical embodiments of the disclosure. However, the disclosure can be implemented in many different forms and is not limited to the embodiments described herein. On the contrary, the embodiments are provided to make the disclosure more thorough and comprehensive.

Unless otherwise defined, all technical and scientific terms used herein have the same meaning as commonly understood by one of ordinary skill in the art to which this disclosure belongs. The terms used in the specification of the disclosure herein are for the purpose of describing particular embodiments only rather than limiting the disclosure.

The present disclosure addresses the issues associated with existing noise-reducing air passages used in PAP machines that rely on foam for noise reduction. These issues include foam degradation, potential health risks to patients, complex manufacturing processes, and environmental concerns. This disclosure provides a safer, more reliable, and structurally simpler noise-reducing air passage device. The noise-reducing air passage device provided by this disclosure overcomes the disadvantages of existing technologies by incorporating noise-reducing components within the air passage device, achieving regulatory noise levels without using foam. This disclosure benefits patients, manufacturers, and the market by providing an advanced technical solution that is also environmentally sustainable.

Detailed embodiments are presented below to elucidate the configurations of the noise-reducing air passage device for use in a PAP machine.

### Embodiment 1

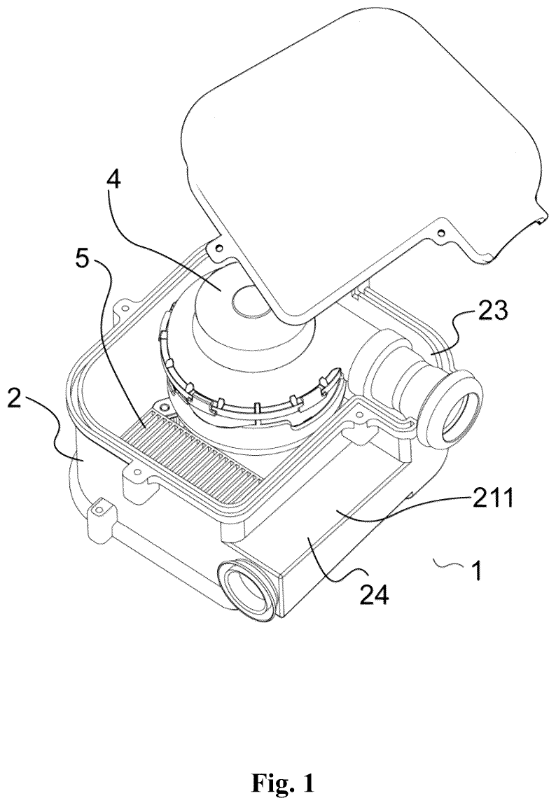

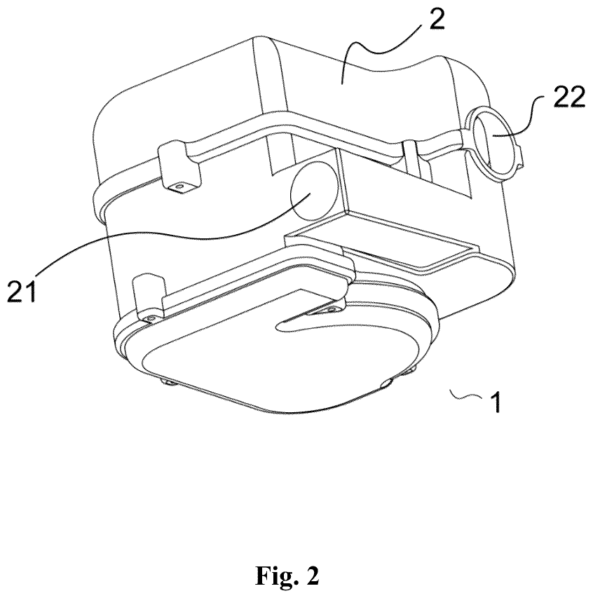

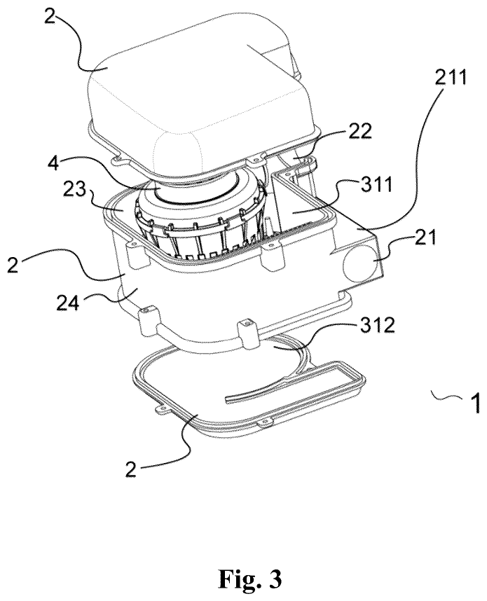

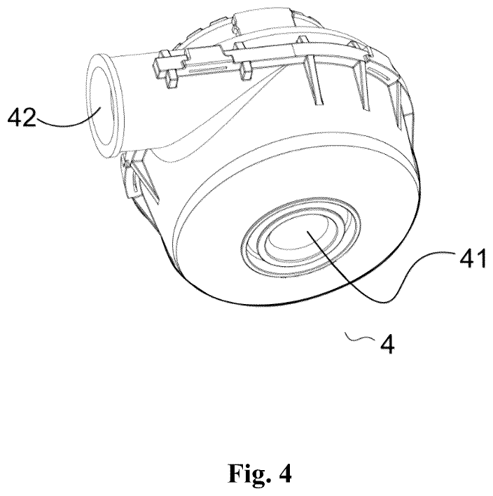

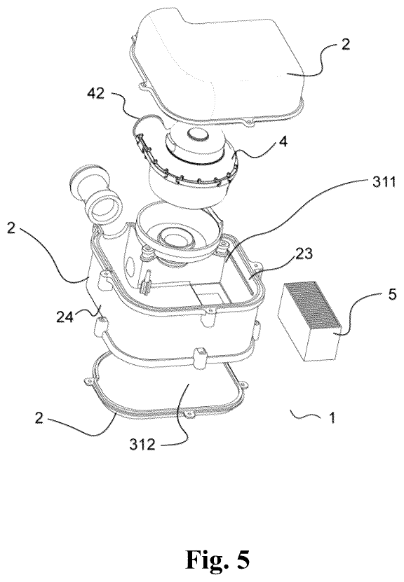

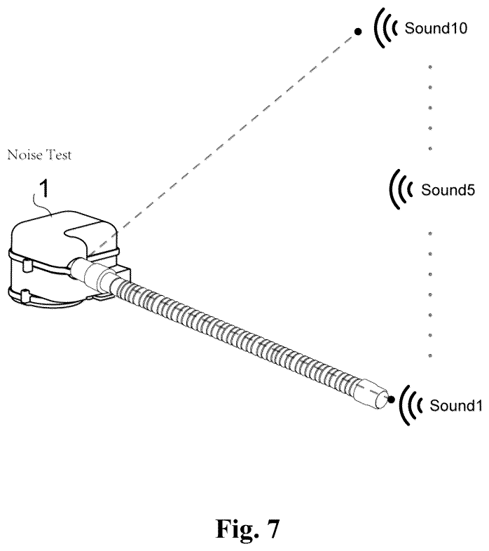

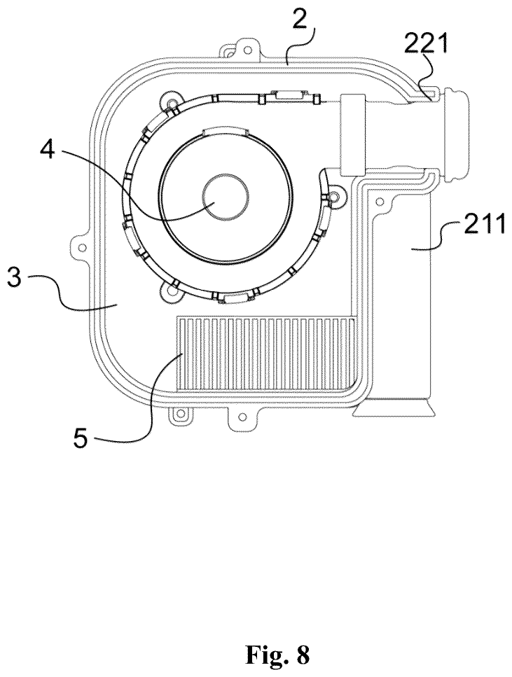

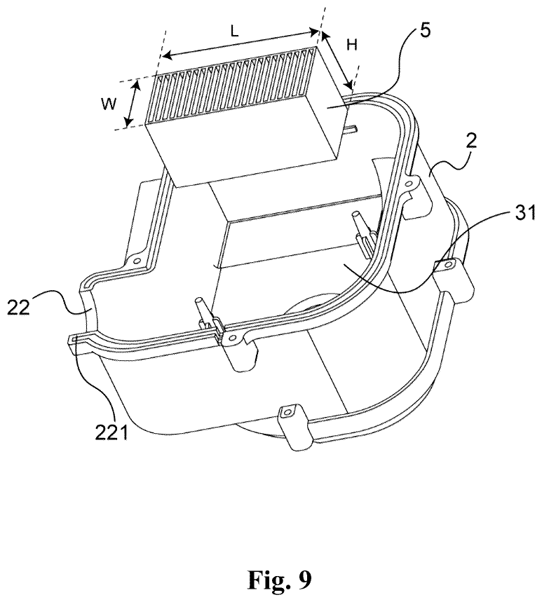

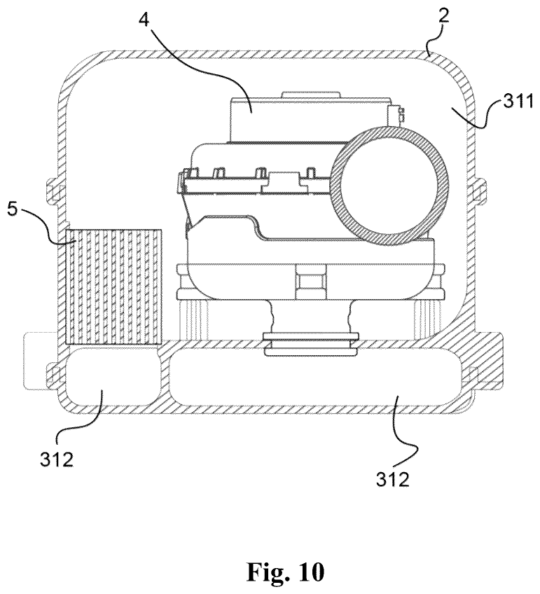

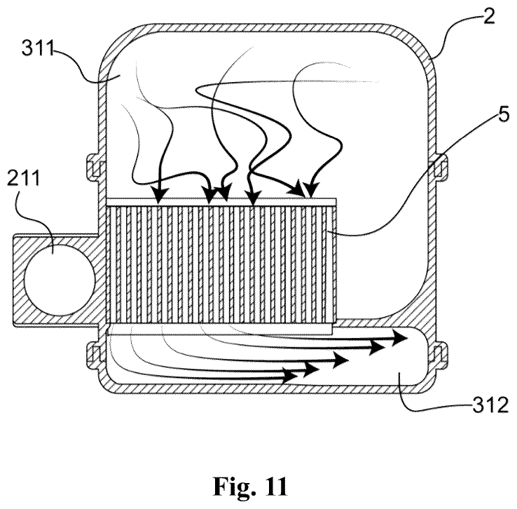

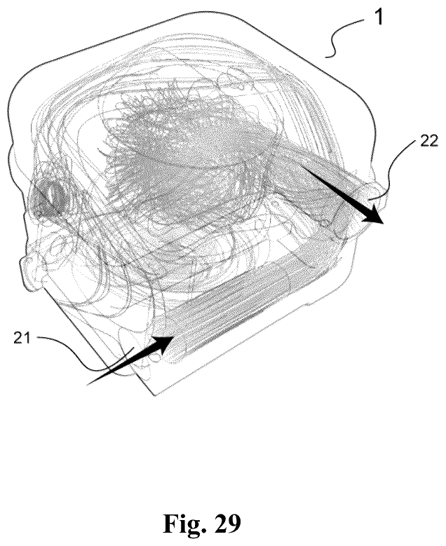

This embodiment provides a noise-reducing air passage device 1 for use in a PAP machine. It includes three-dimensional structural diagrams, exploded views, airflow path diagrams, test scenario diagrams, sectional views, and various data diagrams, as referenced in FIGS. 1-21 and 29. This embodiment involves a noise-reducing air passage device 1 which includes a casing 2 with at least two parts. When assembled into a complete noise-reducing air passage device 1, the casing 2 creates an enclosed space forming a gas passage 3, defined by the inner wall 23. The gas passage 3 is often divided by the structure of the device 1 into chambers 31 and passages through which gas moves from one chamber 31 to another. In some configurations, the gas passage 3 is divided into two or more chambers 31. The noise-reducing air passage device 1 also includes a blower 4 with an intake port 41 and an exhaust port 42 for gas intake and output, respectively. In some instances, the device 1 includes an inlet pipe 211 and an outlet pipe 221, along with noise-reducing components such as a ventilation component 5.

Specifically, the noise-reducing air passage device 1 includes a casing 2 with at least two parts, with at least one outlet 22, at least one inlet 21, an inner wall 23, and an outer wall 24. The inlet 21, which serves as an opening to receive gas into the chamber 31, can take a form of a single or multiple openings without affecting its function. The outlet 22 is configured to communicate with the blower exhaust end 42 on one end and a hose on the other. Typically, there is only one opening as the outlet 22 for the device 1, but the outlet 22 can also take the form of multiple openings in special circumstances. In this embodiment, the outlet 22 and inlet 21 are not on the same wall of the casing 2. Since the noise level at the inlet 21 is typically higher, positioning the inlet 21 and outlet 22 on non-coincident planes helps to prevent noise superposition. A significant distance between the inlet 21 and outlet 22 in the noise-reducing air passage device 1 facilitates noise reduction, thereby lowering the noise levels generated during operation of the device 1.

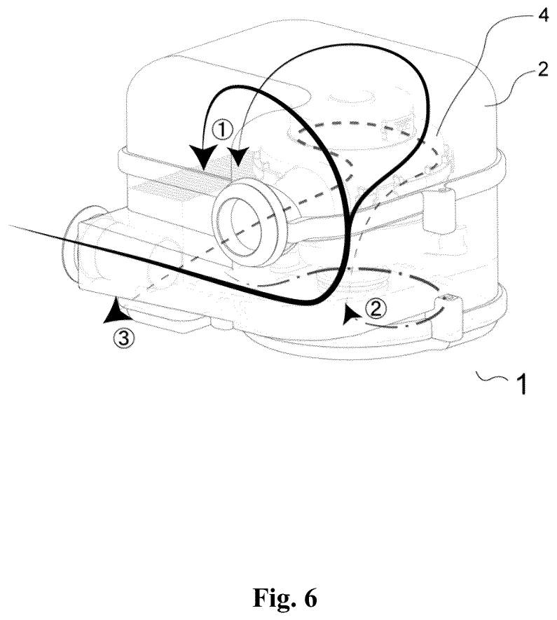

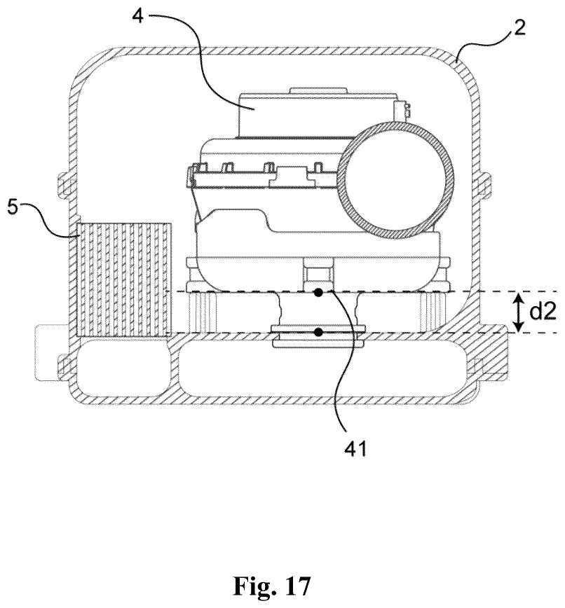

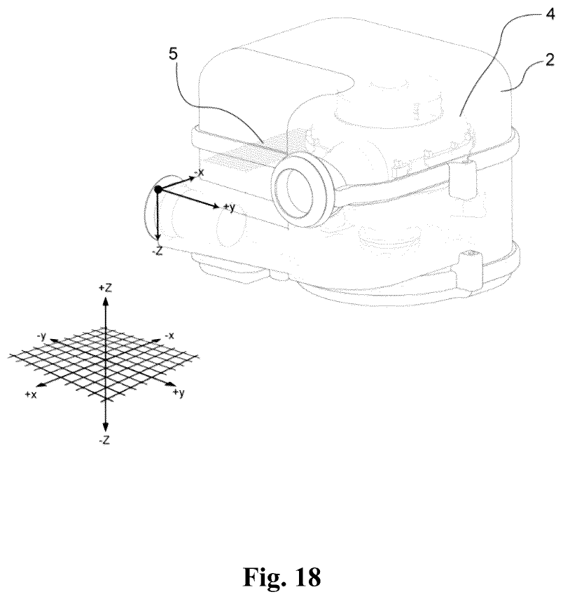

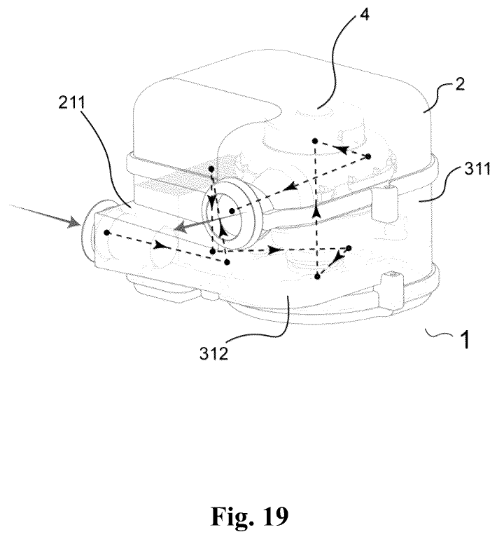

When the casing 2 forms a complete noise-reducing air passage device 1, it creates a gas passage 3 formed by space enclosed by the inner wall 23 of the casing. In this embodiment, there is no foam 6 within gas passage 3. A main path of the gas within the gas passage 3 is an airflow path, and the inner wall opposite the airflow path has an arcuate surface (which is more rounded in shape, this part of the inner wall 23 has continuous curvature rather than angular surfaces). This design reduces friction and resistance within gas passage 3 and facilitates smoother gas flow. This design also plans the airflow path through the configuration of the arcuate surface to prevent abrupt directional changes or obstructions as gas travels through the gas passage 3. The inner wall 23 forms a chamber 31 for gas accumulation within the gas passage 3. The formation of chamber 31 slows down the airflow, aiding in a more stable and smooth gas flow, thus reducing potential turbulence and noise. Another crucial function of the chamber 31 is to provide space to house and secure the blower 4, a core component of noise-reducing air passage device 1, provided within the chamber 31 and configured to pressurize gas entering the chamber 31 and deliver the pressurized gas to the outlet 22 of the casing 2. The blower 4 has an intake port 41 to receive gas and an exhaust port 42 to allow gas to exit. Extensive research and testing have determined that the optimal volume ratio of the chamber 31 to the blower 4 is between 3 to 18 times for better noise reduction of the device 1. The noise-reducing air passage device 1 includes an inlet 21, with the area of the inlet 21 on the casing 2 configured to be equal to or greater than the area of the blower intake port 41, ensuring adequate gas flow into the internal passages of the blower 4. The path of gas through noise-reducing air passage device 1 involves entering the chamber 31 via the inlet 21, flowing through the gas passage 3, entering the blower 4 through the intake port 41 (as illustrated in FIG. 6, parts \textcircled{1} and \textcircled{2}, and exiting through the exhaust port 42 to the outlet 22 (where the pressurized gas generated by the device 1 is discharged, as shown in FIG. 6, part \textcircled{3}). In this disclosure, the gas path within noise-reducing air passage device 1 involves at least two turns, and the path within gas passage 3 includes movement along the x, y, and z axes in a three-dimensional Cartesian coordinate system, with at least three elevation changes, thereby extending the vertical airflow path, where the shortest elevation drop is at least 15 mm. The total length of the airflow path exceeds 20 cm, ideally between 20 cm to 80 cm, with a preferred range of 20 cm to 40 cm (as depicted in FIGS. 18 and 19). This range is determined by the optimal volume ratio of the chamber 31 to the blower 4 being between 3 to 18 times, calculated by the sum of straight lines' length connecting the turning points (if a turning point is an opening, then the center of the opening, such as the center of the inlet 21, the intake end center of the ventilation component 5, and the center of the blower intake port 41). In some configurations, the inner wall 23 forms at least two chambers 31 for gas accumulation within the gas passage 3, aside from the chamber 31 housing the blower 4, with a possible second chamber 312 where gas accumulates smoothly before entering the blower 4 through the intake port 41. In one case, the inner wall 23 forms at least two chambers 31 within the gas passage 3. The at least two chambers include a first chamber 311 and a second chamber 312, with the blower 4 provided in the first chamber 311 and the blower intake port 41 communicating with the second chamber 312, and the second chamber 312 being smaller than the first chamber 311. The gas smoothly gathers in the second chamber 312 before entering the blower 4 through the intake port 41. Additionally, the distance between the blower intake port 41 and the inner wall 23 of the casing 2 is set to be at least 5 mm (as shown in FIG. 17, d2≥5 mm), given the uniform distribution and stability of airflow. Such a distance ensures that airflow entering the blower 4 does not encounter excessive resistance or sudden changes in speed, thus reducing potential turbulence during gas flow, effectively lowering noise levels during airflow. The distance from the blower intake port 41 to its opposing inner wall 23 of the casing 2 is at least partially greater than or equal to 2 mm. Maintaining a certain distance between the inner wall 23 and the blower intake port 41 reduces the resistance faced by gas entering the blower 4, thereby reducing sudden changes in airflow speed and the formation of vortices. This design helps stabilize airflow, effectively reducing noise and vibrations during airflow.

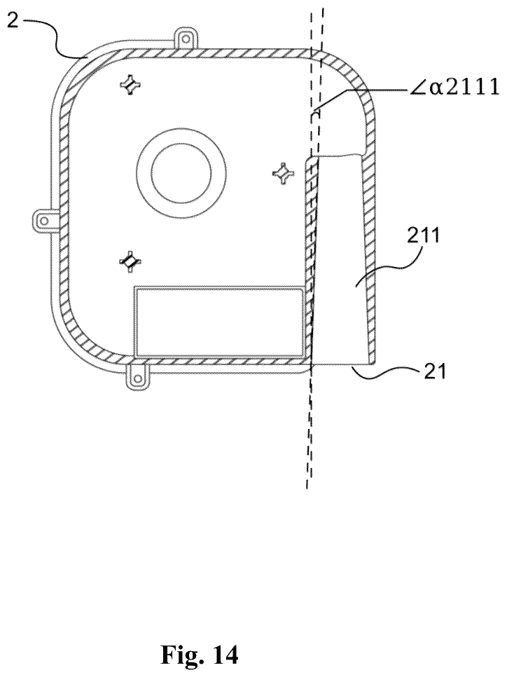

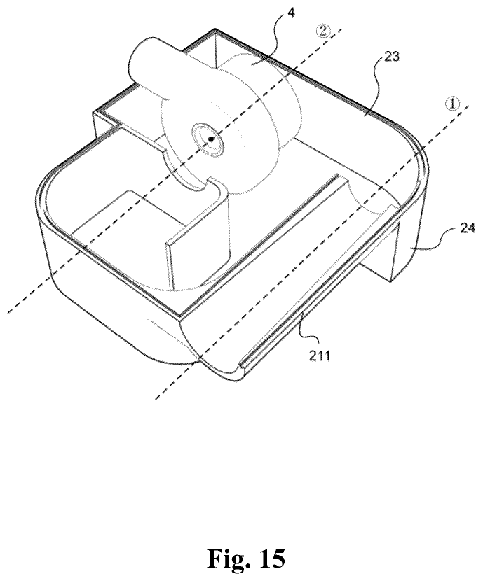

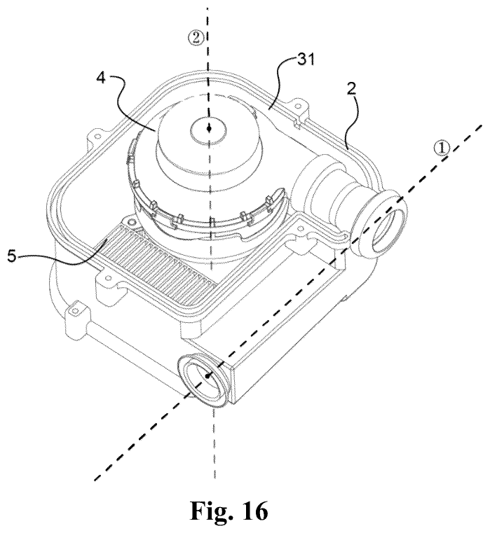

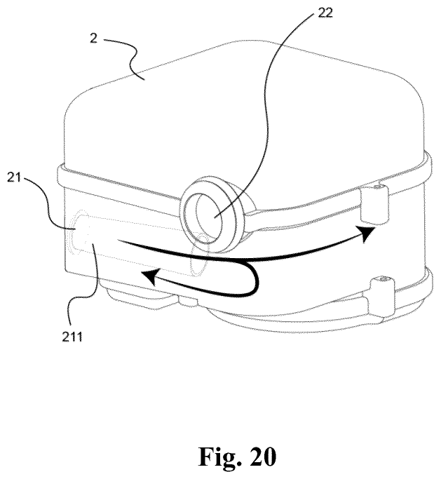

The noise-reducing air passage device 1 also includes at least one inlet pipe 211 provided at and connected to the inlet 21, configured to transport environmental air into the chamber within the casing 2. The connection between the inlet pipe 211 and the casing 2 can be integrally formed or can involve the inlet pipe 211 as a separate component that connects directly or indirectly to the inlet 21 on the casing 2. Due to structural constraints within the noise-reducing air passage device 1, as shown in FIG. 16, the axis of the blower intake port 41 is non-parallel to the axis of the inlet 21 on the casing 2, such as being perpendicular (as shown in FIG. 15, it can also be parallel in another embodiment). In this embodiment, the inlet pipe 211 has a taper 2111 that uses a gradually narrowing cross-section of the passage to streamline the airflow entering the chamber 31 from the inlet 21, allowing the airflow to enter the chamber 31 more smoothly and evenly (as shown in FIG. 14). This design helps reduce the resistance of the airflow entering the chamber 31, thereby reducing noise generation. Meanwhile, the inlet pipe 211 can also enhance transmission efficiency and improve airflow characteristics. In some cases, the inlet pipe 211 is a straight tube without a taper. In some instances, the inlet pipe 211 is provided at the edge portion of the casing 2 (far from the center point of the noise-reducing air passage device 1 and closer to the external environment), making the most of the space within the casing 2 and making the entire gas passage 3 more compact. The form which isolates the inlet pipe 211 from the chamber 31 also effectively directs the path of the gas within gas passage 3, such as some gas flowing towards the gap between the inlet pipe 211 and the wall of casing 2 (as shown in FIG. 20). The inlet pipe 211 can be integrally formed with the casing 2 or connected to it through physical or chemical means. In addition, an outlet pipe 221 is provided at the outlet 22 and communicates with the blower exhaust port 42. The outlet pipe 221 is configured to connect to the blower exhaust port 42, and can be integrally formed with the casing 2. In some implementations, the outlet pipe 221 can also be a separate component connected to the outlet 22 of the casing 2. The end of the outlet pipe 221 nearer the blower 4 is configured to communicate with the blower exhaust port 42; this communication means that the end of the outlet pipe 221 nearer the blower 4 can be directly attached to the blower exhaust port 42 or connected through one or more connecting components.

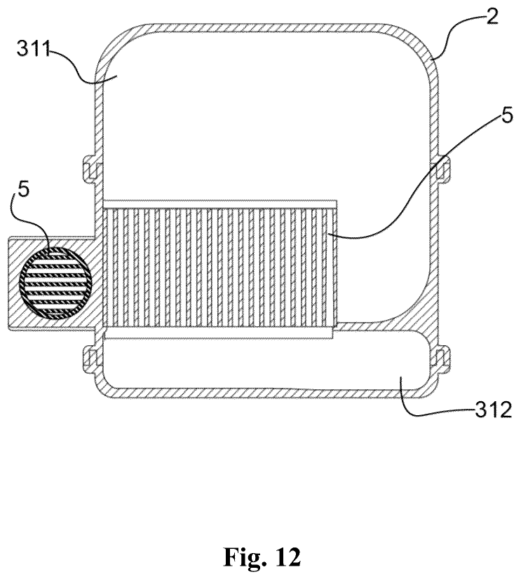

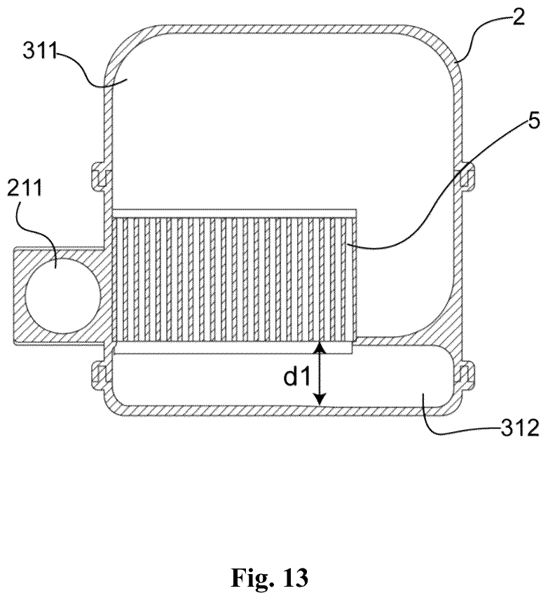

The noise-reducing air passage device 1 also includes at least one ventilation component 5 provided within gas passage 3, configured to communicate with the chambers 31 and provide a channel for gas to flow from one chamber 31 to another. The ventilation component 5 is specifically designed with internal gaps, whereby the airflow is divided as it passes through, and the divided smaller air streams exit through the internal gaps within the ventilation component 5. This configuration of the ventilation component 5 streamlines the otherwise chaotic airflow in gas passage 3, effectively reducing noise by at least 1.5 decibels. Specifically, the ventilation component 5 includes multiple parallel baffles with gaps between them, which are the internal gaps of the ventilation component 5. The ventilation component 5 can take various forms; typically, the baffles are elongated, and their sharp edges help better divide the airflow. In some instances, the baffles may also expand outward or taper inward, accelerating or decelerating the airflow as it passes through the gaps of the ventilation component 5. The ventilation component 5 may also include parallel baffles at two or more angles, intersecting to form channels that divide the airflow. To ensure that the gaps of the ventilation component 5 allow gas to pass through easily and noiselessly, the gaps have a width between 0.8 mm to 2.2 mm. The ventilation component 5 has an intake end and an exhaust end. To provide sufficient space for gas to enter the ventilation component 5, the distance between the exhaust end of the ventilation component 5 and its opposing inner wall 23 of the casing 2 is at least 3.5 mm (as shown in FIG. 13, d1). In other cases, multiple ventilation components 5 with the same form or different forms within the gas passage 3 cooperate to reduce noise (as shown in FIG. 12). Specifically, ventilation components 5 with the same form or different forms (e.g., with different baffle angles or tapered baffles) are provided within gas passage 3, tightly fitting against each other, with their internal gaps interconnected. In some instances, ventilation components 5 with the same form or different forms are placed at various locations within the gas passage 3. In some embodiments, the ventilation component 5 is integrally formed with the gas passage 3.

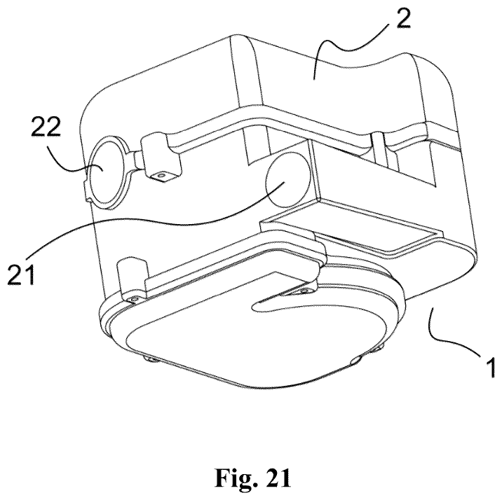

In another embodiment, the outlet 22 and inlet 21 are on the same wall of the casing 2 (as shown in FIG. 21).

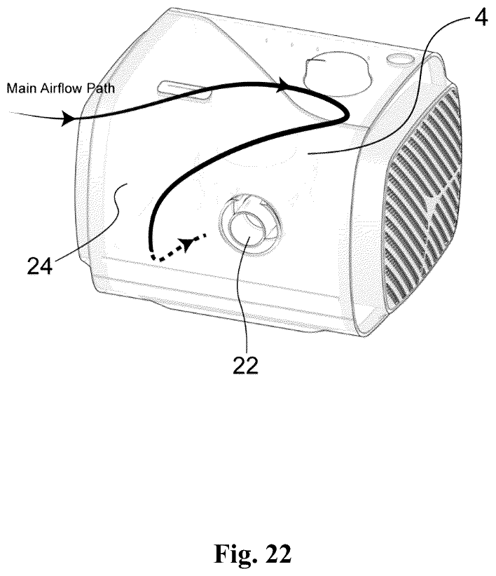

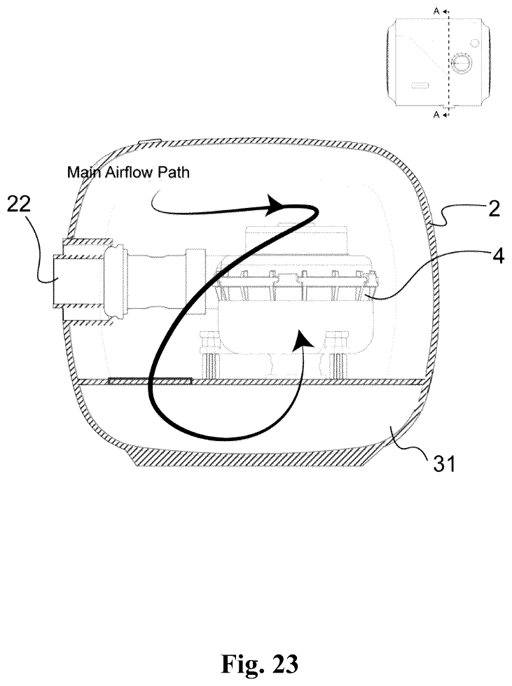

In another embodiment, the casing 2 forms part of a PAP machine (as shown in FIGS. 22 and 23), where the blower 4 is at least partially exposed inside the casing of the PAP machine, meaning the casing 2 of the noise-reducing air passage device 1 is at least partially the same part as the casing of the PAP machine.

### Embodiment 2

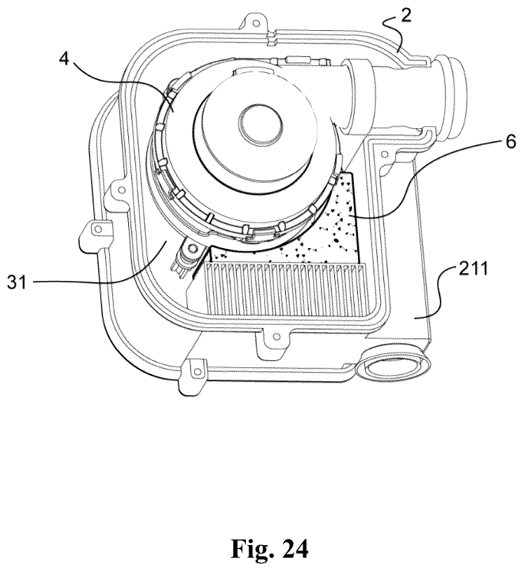

This embodiment provides a noise-reducing air passage device 1 for use in a PAP machine, as shown in FIG. 24. This embodiment provides a structural diagram of noise-reducing air passage device 1. The difference in this embodiment from Embodiment 1 is that the chamber 31 contains foam 6. The noise-reducing air passage device 1 provided by this disclosure can meet regulatory noise levels even without foam 6, thus accommodating different patient needs for noise reduction or safety. Some patients may prioritize the health impacts of the PAP machine, and thus may choose a noise-reducing air passage device 1 without foam 6, eliminating potential health risks from the foam material. Other patients may focus more on the quietness of the PAP machine. With proper health considerations, foam 6 can be added to the noise-reducing air passage device 1 of this disclosure to achieve an even quieter operation, surpassing the quietness of existing market PAP machines, further reducing noise beyond the regulatory levels achieved without foam 6. This design creates a quieter, more comfortable environment, fulfilling the personalized needs of different patients.

### Embodiment 3

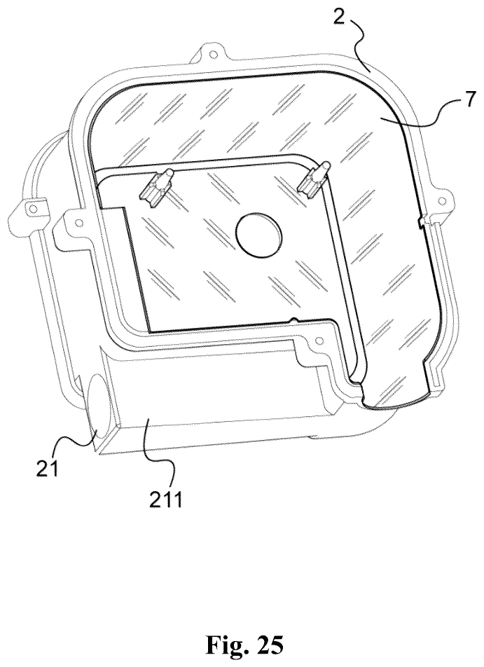

This embodiment provides a noise-reducing air passage device 1 for use in a PAP machine, as referenced in FIG. 25. It offers a schematic diagram of the noise-reducing air passage device 1. The difference of this embodiment from Embodiment 1 is that the inner wall 23 of the casing 2 has a silicone layer 7 connected to the inner wall, configured to reduce noise within gas passage 3. Firstly, silicone material is selected for its excellent sound absorption and insulation properties, effectively mitigating the noise generated by gas flowing through the noise-reducing air passage device 1, thus lowering the overall noise level of the machine. The silicone material used in this embodiment is soft and elastic, allowing it to absorb vibrations caused by airflow effectively, thereby preventing noise transmission outside of the noise-reducing air passage device 1. Secondly, the soft and smooth surface of the silicone material covers the inner wall 23 of the casing 2, forming a buffer layer that is heat-resistant and wear-resistant, which maintains stable performance of the PAP machine over long-term use and extends the machine's lifespan. Its durability and stability ensure that the PAP machine can maintain effective noise reduction performance over time, providing a consistently stable breathing environment for patients. Moreover, silicone material boasts excellent biocompatibility, is harmless to humans, and does not cause allergies or adverse reactions, making it safe for use in PAP machines in direct contact with patients. The use of this material further enhances the safety and comfort of the PAP machine, ensuring that patients experience no adverse effects during use, thereby improving patient satisfaction.

In another embodiment, other materials with soundproofing or sound-absorbing properties could also be applied to the inner wall 23 of the casing 2 in the noise-reducing air passage device 1 to achieve noise reduction.

### Embodiment 4

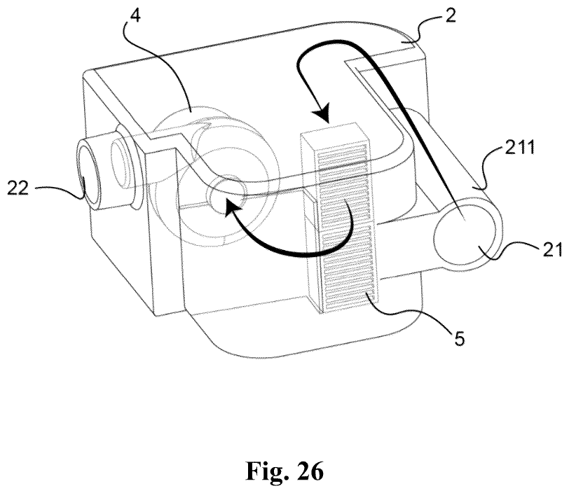

This embodiment introduces a noise-reducing air passage device 1 for use in a PAP machine, as shown in FIG. 26. It provides a schematic diagram of the noise-reducing air passage device 1. The difference in this embodiment from Embodiment 1 is the positioning of the blower 4 within the device 1. Specifically, the airflow path within noise-reducing air passage device 1 takes a basic horizontal form, where the airflow largely remains on the same horizontal plane, with limited or no vertical pathways (the vertical pathway length being less than 40 mm). And the chamber 31 of the noise-reducing air passage device 1 is divided left-to-right or front-to-back rather than top-to-bottom. The airflow within gas passage 3 follows a roughly S-shaped path, and in the top view, the airflow path shows no significant overlaps. The noise reduction structures or components of this disclosure can be placed into different configurations of the noise-reducing air passage device 1 to achieve noise reduction.

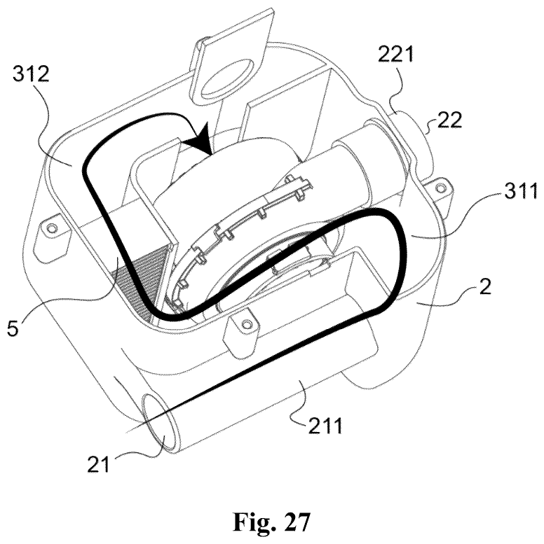

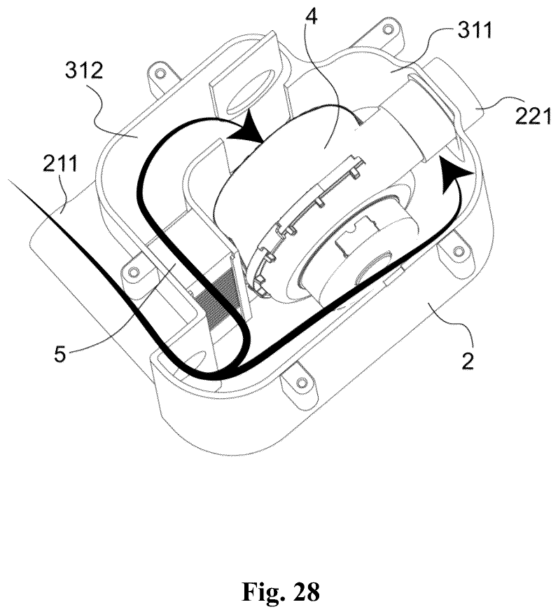

In another embodiment, two different structural configurations of the noise-reducing air passage device 1 derived from Embodiment 4 are developed, the axis of the inlet pipe 211 is perpendicular or parallel to the axis of blower 4, as shown in FIGS. 27 and 28.

The implementation of the noise-reducing air passage device provided by this disclosure has at least the following beneficial effects:

1. Ventilators must achieve a noise level of 30 dB or less for marketing approval in FDA's regulations. Using foam for noise reduction is currently the simplest method. Foam materials, which are readily available and easy to manufacture, can convert noise into minute amounts of energy due to their unique porous structure and material properties. Indeed, using foam for noise reduction achieves effective noise mitigation and the use of foam within the air passage device is the most straightforward and common method to meet regulatory noise levels.

Consequently, nearly all PAP machines on the market contain foam within the gas passage device for noise reduction. However, foam can easily cause health issues for several reasons: 1) Foam materials are soft and have a relatively loose surface, which can wear or peel off during use, releasing particles. Once these particles are released, they can enter the patient's respiratory tract with the airflow, causing irritation to the respiratory system. This may lead to respiratory issues, resulting in symptoms such as sore throat and coughing, especially in individuals who already suffer from asthma or Chronic Obstructive Pulmonary Disease (COPD). 2) Moreover, foam is usually made from synthetic materials that may contain chemical additives. These chemicals can gradually release into the air as the foam ages. In some cases, if foam particles carry harmful microorganisms, they could cause potential infections, particularly in individuals with compromised immune systems. Foam particles may also trigger allergic reactions, including sneezing, flu-like symptoms, and eye irritation. 3) Additionally, foam used over long periods can accumulate dust, bacteria, and other contaminants. This is particularly concerning in PAP machines, where the foam can draw pollutants from the air into the machine, fostering bacterial growth and increasing the risk of infection.

Therefore, the disclosure particularly focuses on the safety and reliability of air passage devices of the ventilator during design, involving a series of improvements, including a foam-free design for the noise-reducing air passage device, to reduce potential health risks for patients using PAP machines. The gas passage and filtration system are designed foam-free or using easily replaceable foam to reduce these potential health risks. For health and safety, the gas passage in the disclosure is designed without foam. Since there is no foam within the gas passage, the likelihood of accumulating small foreign particles is reduced, helping to maintain the cleanliness of the gas passage. More importantly, in the absence of foam, the breathable air will not be affected by the tiny residues of the foam, reducing the number of particles that patients might inhale or come into contact with, thus ensuring safety during use. This is especially important for patients who use the device long-term, as it helps to reduce potential respiratory issues. Additionally, some patients may be allergic to particles from materials such as foam, and the foam-free design reduces the risk associated with allergic reactions. This is particularly significant for patients who are allergic to foam materials or sensitive to chemically treated materials. This product has undergone multiple tests to verify that the foam-free noise-reducing air passage device improves the safety and comfort of patients using the device.

2. The design of various noise-reducing components and the internal space structure of the noise-reducing air passage device ensures compliance with regulatory noise levels, even in the absence of foam. 1) This disclosure uses various types of noise-reducing components and structures, which combined, form an efficient noise-reduction system achieving significant noise mitigation. a. The internal space structure of the noise-reducing air passage device is configured to extend the airflow path within the gas passage. By lengthening the airflow path, the residence time of the gas within the gas passage increases, helping to reduce the speed of the airflow and the potential for turbulence or vortices, thereby lowering the noise level. A longer airflow path also means that the gas travels a greater distance within the gas passage, allowing noise to attenuate further during transmission. Therefore, extending the airflow path can increase the attenuation distance of noise, making it weaker by the time it reaches the designated position. b. The disclosure also introduces a ventilation component within the gas passage for noise reduction, an innovative structure not found in existing PAP machines' gas passages. The ventilation component effectively reduces turbulence and vortices in the airflow, thereby lowering noise levels. Additionally, the ventilation component offers advantages over existing noise-reducing components in PAP machines on the market. The ventilation component is simple in structure, integrally molded from a single material, eliminating unnecessary steps in manufacturing and installation, thereby reducing costs. Moreover, it can be individually optimized with different forms of microstructural variations, including changes to its baffle design. Due to its simple structure, the ventilation component can be installed at various positions within the gas passage for noise reduction. The noise-reducing air passage device can also include a combination of ventilation components of the same or different forms, providing greater flexibility in its application across different noise-reducing air passage devices. The core of the ventilation component lies in its internal structural design, allowing it to maintain its structure while adapting its external shape to fit different types of noise-reducing air passage devices or be placed in various positions within the device, such as inside the inlet pipe. This flexible design allows the ventilation component to be customized for specific applications to achieve better noise reduction. Regardless of the type of noise-reducing air passage devices, the ventilation component can maintain its noise-reducing performance, providing an efficient noise control solution for PAP machines. c. An inlet pipe is set at the inlet. Through the inlet pipe, external air can enter the chamber in a more orderly manner, reducing potential turbulence and noise generated when the airflow enters the chamber. Additionally, the inlet pipe stabilizes the airflow path within the chamber, reducing resistance as the air enters, further decreasing noise. The inlet pipe also enhances the efficiency of air transmission into the chamber, improving airflow characteristics and enhancing the overall performance of the PAP machine. d. The arrangement of the inlet and outlet not being on the same wall of the casing typically helps in noise reduction. Normally, the inlet is one of the primary sources of noise inside a PAP machine. Placing the inlet and outlet on different walls of the casing can prevent noise accumulation at the inlet, thus reducing noise levels at the inlet and enhancing the overall noise reduction effect. The combination of the above noise-reducing structures and components allows the noise-reducing air passage device to meet regulatory noise levels without foam and is a more effective and safer new noise reduction method. 2) By employing the above design of the noise-reducing air passage device, the disclosure achieves regulatory noise levels without foam. When foam is introduced within the noise-reducing air passage device provided by this disclosure, it can achieve even higher noise reduction levels compared to the foam-free ones. Therefore, while ensuring safety, the introduction of foam or other soundproofing materials (such as silicone, gel, etc.) within the gas passage increases the absorption and isolation of noise in the airflow, further reducing the propagation and impact of noise. Compared to the foam-free situation, a noise-reducing air passage device with foam can provide a quieter environment, offering patients a more comfortable experience. This design retains the noise reduction advantages of the foam-free condition while further enhancing the noise reduction effect, achieving or even surpassing the highest noise reduction levels of existing PAP machines on the market, thus meeting the demand for higher noise reduction performance.

3. The noise-reducing air passage device provided by this disclosure has a simple structure and modular advantages, making it a cost-effective solution for manufacturers. The simple structure of the ventilation component allows for easy assembly or standalone use, enhancing efficiency during manufacturing, assembly, and maintenance. The modular approach enables different parts to be independently manufactured and upgraded, improving production efficiency and reducing costs. Manufacturers can customize the noise-reducing air passage devices by assembling components to meet various patient needs or product specifications. This economic design reduces manufacturing costs and offers patients more flexible and affordable options. Since the primary goal of this disclosure is to achieve regulatory noise levels without foam, there is no need to consider the layout and installation of foam. Consequently, noise-reducing air passages without foam have a simpler internal structure compared to existing market options. The simplified structure enhances the efficiency of manufacturing and assembly processes, reducing production costs and cycles.

The above description of the embodiments of the disclosure is provided with reference to the accompanying drawings. However, the disclosure is not limited to the specific embodiments described above. These specific embodiments are merely illustrative and not restrictive. Those skilled in the art, in light of the teachings of the disclosure, may make many modifications and variations without departing from the spirit and scope of the disclosure as defined by the claims. All such modifications and variations are within the protection scope of the disclosure.

It must be noted that as used herein and in the appended claims, the singular forms “a”, “an”, and “the” include their plural equivalents, unless the context clearly dictates otherwise.

1. A noise-reducing air passage device for use in a PAP machine, configured to generate and deliver pressurized gas to an airway of a patient, the noise-reducing air passage device comprising:
a casing comprising at least two parts, having an outlet, an inlet, inner walls, and outer walls;
a gas passage, formed by a space enclosed by the inner walls of the casing, wherein the gas passage comprises at least one chamber configured to provide a space for gas accumulation and flow, and wherein a total volume of the at least one chamber is between 3 to 18 times a volume of a blower;
the blower, including an intake port to receive gas and an exhaust port to allow the pressurized gas to exit, wherein the blower is provided within one of the at least one chamber, and is configured to pressurize the gas that enters the at least one chamber and deliver the pressurized gas to the outlet of the casing;
wherein an inlet pipe is provided at and connectable to the inlet of the casing, and the inlet pipe is configured to deliver the gas from an external environment into the at least one chamber within the casing;
wherein the outlet and the inlet are not provided on a same inner wall or outer wall of the casing;
wherein the casing is configured such that a path of the gas includes the gas entering the at least one chamber via the inlet of the casing, flowing through the gas passage, entering the blower through the intake port, and exiting the casing through the outlet, wherein the path of the gas includes at least two turns and movement along x, y, and z axes in a three-dimensional Cartesian coordinate system and at least three elevation changes along the z-axis from the gas entering and the gas exiting the casing,
wherein the at least three elevation changes include at least an elevation change within the gas passage from above the at least two chambers into the intake port of the blower provided within one of the at least two chambers,
wherein a shortest vertical elevation drop of the path of the gas along the z-axis is at least 15 mm, and
wherein the at least one chamber does not include foam.

2. The noise-reducing air passage device according to claim 1, wherein the inlet pipe is configured to be integrally formed with the casing.

3. The noise-reducing air passage device according to claim 1, wherein a distance between the intake port of the blower and the inner walls of the casing is at least 5 mm.

4. The noise-reducing air passage device according to claim 1, wherein an axis of the inlet pipe is parallel or perpendicular to an axis of the intake port of the blower.

5. The noise-reducing air passage device according to claim 1, wherein an outlet pipe is provided at the outlet and communicates with the exhaust port of the blower.

6. The noise-reducing air passage device according to claim 1, wherein the casing forms a part of the PAP machine.

7. A noise-reducing air passage device for use in a PAP machine, configured to generate and deliver pressurized gas to an airway of a patient, the noise-reducing air passage device comprising:
a casing comprising at least two parts, having an outlet, an inlet, inner walls, and outer walls;
a gas passage, formed by a space enclosed by the inner walls of the casing, wherein the gas passage comprises at least two chambers configured to provide a space for gas accumulation and flow, and wherein a total volume of the at least two chambers is between 3 to 18 times a volume of a blower;
the blower, including an intake port to receive gas and an exhaust port to allow the pressurized gas to exit, wherein the blower is provided within one of the at least two chambers, and is configured to pressurize the gas that enters the at least two chambers and deliver the pressurized gas to the outlet of the casing;
wherein an inlet pipe is provided at and connectable to the inlet of the casing, and the inlet pipe is configured to deliver the gas from an external environment into the at least two chambers within the casing;
wherein the casing is configured such that a path of the gas includes the gas entering the at least two chambers via the inlet of the casing, flowing through the gas passage, entering the blower through the intake port, and exiting the casing through the outlet, wherein the path of the gas includes at least two turns and movement along x, y, and z axes in a three-dimensional Cartesian coordinate system and at least three elevation changes along the z-axis from the gas entering and the gas exiting the casing,
wherein the at least three elevation changes include at least an elevation change within the gas passage from above the at least two chambers into the intake port of the blower provided within one of the at least two chambers, and
wherein the at least two chambers do not include foam.

8. The noise-reducing air passage device according to claim 7, wherein the inner walls of the casing include a silicone layer connectable to the inner walls, and the silicone layer is configured to reduce noise within the gas passage.

9. The noise-reducing air passage device according to claim 7, wherein the inlet pipe is provided at an edge portion of the casing, and the inlet pipe and the outlet of the casing are not on a same wall of the casing.

10. The noise-reducing air passage device according to claim 7, wherein the inlet pipe is tapered.

11. The noise-reducing air passage device according to claim 7, wherein a distance between the intake port of the blower and its opposing inner wall of the casing is at least 2 mm.

12. The noise-reducing air passage device according to claim 7, wherein the casing forms a part of the PAP machine.

13. A noise-reducing air passage device for use in a PAP machine, configured to generate and deliver pressurized gas to an airway of a patient, the noise-reducing air passage device comprising:
a casing comprising at least two parts, having an outlet, an inlet, inner walls, and outer walls;
a gas passage, formed by a space enclosed by the inner walls of the casing, wherein the gas passage comprises at least one chamber configured to provide a space for gas accumulation and flow, and wherein a total volume of the at least one chamber is between 3 to 18 times a volume of a blower;
the blower, including an intake port to receive gas and an exhaust port to allow the pressurized gas to exit, wherein the blower is provided within one of the at least one chamber, and wherein the blower is configured to pressurize the gas that enters the at least one chamber and deliver the pressurized gas to the outlet of the casing;
wherein an inlet pipe is provided at and connectable to the inlet of the casing, and the inlet pipe is configured to deliver the gas from an external environment into the at least one chamber within the casing;
wherein the casing is configured such that a path of the gas includes the gas entering the at least one chamber via the inlet of the casing, flowing through the gas passage, entering the blower through the intake port, and exiting the casing through the outlet, wherein the path of the gas includes at least two turns and movement along x, y, and z axes in a three-dimensional Cartesian coordinate system and at least three elevation changes along the z-axis from the gas entering and the gas exiting the casing,
wherein the at least three elevation changes include at least an elevation change within the gas passage from above the at least one chamber into the intake port of the blower provided within one of the at least one chamber, and
wherein an axis of the intake port of the blower is non-parallel to an axis of the inlet of the casing.

14. The noise-reducing air passage device according to claim 13, wherein the inner walls of the casing include a silicone layer connectable to the inner walls, and the silicone layer is configured to reduce noise within the gas passage.

15. The noise-reducing air passage device according to claim 13, wherein the inner walls of the casing are configured to form at least two chambers for gas accumulation within the gas passage.

16. The noise-reducing air passage device according to claim 13, wherein the inner walls of the casing opposite the path of the gas have an arcuate surface.

17. The noise-reducing air passage device according to claim 13, wherein a distance between the intake port of the blower and its opposing inner wall of the casing is at least 2 mm.

18. The noise-reducing air passage device according to claim 13, wherein the casing forms a part of the PAP machine.

19. The noise-reducing air passage device according to claim 13, wherein the at least one chamber does not include foam.

20. A noise-reducing air passage device for use in a PAP machine, configured to generate and deliver pressurized gas to an airway of a patient, the noise-reducing air passage device comprising:
a casing comprising at least two parts, having an outlet, an inlet, inner walls, and outer walls;
a gas passage, formed by a space enclosed by the inner walls of the casing, wherein the gas passage comprises at least two chambers configured to provide a space for gas accumulation and flow, and wherein a total volume of the at least two chambers is between 3 to 18 times a volume of a blower;
the blower, including an intake port to receive gas and an exhaust port to allow the pressurized gas to exit, wherein the blower is provided within one of the at least two chambers, and wherein the blower is configured to pressurize the gas that enters the at least two chambers and deliver the pressurized gas to the outlet of the casing;
wherein the noise-reducing air passage device further comprises at least one ventilation component that is provided within the gas passage, configured to communicate with the at least two chambers and provide a channel for the gas to flow from one chamber to another;
wherein a main path of the gas within the gas passage is an airflow path, wherein the airflow path is configured to have at least two turns and displacements along an x-axis, y-axis, and z-axis in a three-dimensional Cartesian coordinate system and at least three elevation changes along the z-axis from the gas entering the blower through the intake port and the gas exiting the outlet of the casing, and a total length of the airflow path is greater than 20 cm, and wherein a shortest vertical elevation drop of the path of the gas along the z-axis is at least 15 mm;
wherein the at least three elevation changes include at least an elevation change within the gas passage from above the at least two chambers into the intake port of the blower provided within one of the at least two chambers, and
wherein an area of the inlet of the casing is greater than or equal to an area of the intake port of the blower.

21. The noise-reducing air passage device according to claim 20, wherein the at least two chambers comprise a first chamber and a second chamber, the blower is provided within the first chamber, and the intake port of the blower communicates with the second chamber.

22. The noise-reducing air passage device according to claim 21, wherein the second chamber is smaller than the first chamber.

23. The noise-reducing air passage device according to claim 20, wherein the ventilation component includes an intake end and an exhaust end, and a distance between the exhaust end and its opposing inner wall of the casing is at least 3.5 mm.

24. The noise-reducing air passage device according to claim 20, wherein an outlet pipe is provided at the outlet and communicates with the exhaust port of the blower.

25. The noise-reducing air passage device according to claim 20, wherein the casing forms a part of the PAP machine.
